(19) United States
(12) Patent Application Publication
(10) Pub. No.: US 2005/0020900 A1
(43) Pub. Date: Jan. 27, 2005

(54) Method and system for measuring in a dynamic sequence of medical images

(75) Inventors: Jonas Yngvesson, Rimforsa (SE); Anders Kahari, Orebro (SE)

Correspondence Address: VENABLE, BAETJER, HOWARD AND CIVILETTI, LLP, P.O. BOX 34385, WASHINGTON, DC, 20043-9998, US

(73) Assignee: Sectra Imtec AB, Linkoping (SE)

(21) Appl. No.: 10/813,555

(22) Filed: Mar. 31, 2004

(30) Foreign Application Priority Data
Apr. 1, 2003 (SE) 0300951-1

(51) Int. Cl. A61B 5/05

(52) U.S. Cl. 600/407; 128/922

(57) ABSTRACT

The present invention relates to a method and a system for measuring in a dynamic sequence of medical images of a moving body part. A reference point being fixed relative to an image geometry and at least one measurement point are defined in the moving body part in one image in the sequence of images. The reference point is then automatically indicated and the at least one measurement point is then automatically tracked in all of the images of the sequence. A length and a direction of at least one vector extending from the reference point to one of the at least one measurement points for each pair of reference point and one measurement point is automatically determined in all of the images of the sequence and at least one of a rate of change of the length and the direction of the at least one vector is automatically determined between selected images in the sequence of images.

### TECHNICAL FIELD

The present invention relates to a method and a system for measuring in a dynamic sequence of medical images of a moving body part.

### BACKGROUND OF THE INVENTION

Non-invasive methods for measuring functions and dysfunctions of body organs typically involve scanning with an X-ray device to obtain projection data for generation of a series of images. When measurements are to be performed of a moving body part, images of several events of the movement have to be created. Images are then generated with a certain frequency, whereby a sequence of consecutive images is formed. The sequence depicts the same anatomical area over a period of time during which some dynamic event takes place and is thus a dynamic sequence. An example of such a dynamic sequence is an image sequence of the heart over one or more heartbeat cycles.

In order to be able to perform measurements in the images, different kinds of measurement tools for application to the images are needed. A fundamental tool in image measurements is the distance measurement tool. Another important tool is the angle measurement tool for measuring the angle between two lines superimposed on an image.

Common for many tools available today is that they measure static data in static images. When a distance measurement is to be done in a static image, a user identifies two anatomical features in the image and applies a distance measurement tool to determine the distance between them.

There are tools and methods for measuring in dynamic sequences, but they consist of two or more instances of an intrinsically static tool that is more or less manually applied to several of the images in a dynamic sequence. The result from these different instances may then be used for comparisons or to compute cruder approximations to dynamic entities by using ratios or differentiations.

For example, a commonly used method today for measuring in a dynamic sequence is a method of distance measurement applied to an image sequence from coronary angiography involving the following steps: manual identification of two images in the sequence, showing, for example, the end-diastole and end-systole phase in the same heart cycle; manual location of the most apical part of the coronary artery branches in both images; manual location of the lower contour of the left coronary ostium in both images; manual location of two points on the horizontal part of the circumflex artery, one proximal and one distal, in both images; measurement of the distance from the point from the second step to the point from the third step in one image and comparison with the distance between the same points in the other image; and measurement of the distance from the point from the second step to the points from the fourth step and comparison with the distance between the same points in the other image.

Performing the above mentioned steps only once gives very limited dynamic information since only two images in the sequence are used for the measurements. To obtain more dynamic information, a user has to repeat the steps for every image in the dynamic sequence or at least for many images in the dynamic sequence. Usually there are over 100 images in a sequence. Thus, using the above mentioned method on all images in an image sequence is rather time-consuming.

Through EP 1 088 517 a method and an apparatus for motion-free cardiac imaging are known. In EP 1 088 517 a fixed reference point and a dynamic point are used for selecting images without motion-induced artefacts in a dynamic sequence of computerized tomographic images. A line is drawn from the reference point to the dynamic point in all of the images. The line length represents the distance between the reference point and the dynamic point and one image or images are selected in which the line length remains constant relative to the previous image.

Image recognition software may be used either to identify a pair of reference points for measurements of relative motion or, once reference points are first identified, to identify corresponding reference points on other images. Distances between the automatically identified points may be selected by software, based upon selected criteria. However, in EP 1 088 517 determination of other variables such as speed, acceleration and retardation are not described for measurement in a dynamic sequence of images of a moving body organ. Furthermore, only two points, one fixed reference point and one dynamic point are used. It is an advantage to be able to use more than two points as well as more than one dynamic point. For example, angle measurements and area measurements require at least three points and measurements of relative motion of two moving parts of a moving body part require at least two dynamic points.

SUMMARY OF THE INVENTION

Accordingly, it is an object of preferred embodiments of the present invention to provide an improved method for measuring in a dynamic sequence of medical images of a moving body part.

This object my be achieved by means of a method for measuring in a dynamic sequence of medical images of a moving body part comprising: defining at least one measurement point in the moving body part in one of said images; defining a reference point in one of said images to a point being fixed relative to the image geometry, said reference point being different from said at least one measurement point; automatically tracking the at least one measurement point in all of said images of the sequence; automatically indicating the reference point in all of said images of the sequence; automatically determining a length and a direction of a vector extending from the reference point to one of the at least one measurement points for each pair of reference point and one measurement point in all of said images of the sequence, and automatically determining a rate of change of said length and/or said direction of said vector(s) between selected images in said sequence of images.

Another object of preferred embodiments of the present invention is to provide an improved method for generating a dynamic sequence of medical images of a moving body part and measuring in said dynamic sequence.

This object may be achieved by means of a method for generating a dynamic sequence of medical images of a moving body part and measuring in said dynamic sequence, which method comprises the steps of: scanning a portion of a body of a patient including the moving body part for generating time resolved projection data; generating said images from said projection data; defining at least one measurement point in the moving body part in one of said images; defining a reference point in one of said images to a point being fixed relative to the image geometry, said reference point being different from said at least one measurement point; automatically tracking the at least one measurement point in all of said images of the sequence; automatically indicating the reference point in all of said images of the sequence; automatically determining a length and a direction of a vector extending from the reference point to one of the at least one measurement points for each pair of reference point and one measurement point in all of said images of the sequence, and automatically determining a rate of change of said length and/or said direction of said vector(s) between selected images in said sequence of images.

A further object of preferred embodiments of the present invention is to provide an improved system for measuring in a dynamic sequence of medical images of a moving body part.

This object may be achieved through a system for measuring in a dynamic sequence of medical images of a moving body part, said system having means for: defining at least one measurement point in the moving body part in one of said images; defining a reference point in one of said images to a point being fixed relative to the image geometry, said reference point being different from said at least one measurement point; automatically tracking the at least one measurement point in all of said images of the sequence; automatically indicating the reference point in all of said images of the sequence; automatically determining a length and a direction of a vector extending from the reference point to one of the at least one measurement points for each pair of reference point and one measurement point in all of said images of the sequence, and automatically determining a rate of change of said length and/or said direction of said vector(s) between selected images in said sequence of images.

Another object of preferred embodiments of the present invention is to provide an improved system for generating a dynamic sequence of medical images of a moving body part and for measuring in said dynamic sequence. This object may be achieved through a system for generating a dynamic sequence of medical images of a moving body part and for measuring in said dynamic sequence, said system having means for: scanning a portion of a body of a patient including the moving body part for generating time resolved projection data; generating said images from said projection data; defining at least one measurement point in the moving body part in one of said images; defining a reference point in one of said images to a point being fixed relative to the image geometry, said reference point being different from said at least one measurement point; automatically tracking the at least one measurement point in all of said images of the sequence; automatically indicating the reference point in all of said images of the sequence; automatically determining a length and a direction of a vector extending from the reference point to one of the at least one measurement points for each pair of reference point and one measurement point in all of said images of the sequence, and automatically determining a rate of change of said length and/or said direction of said vector(s) between selected images in said sequence of images.

Advantages of the methods and the systems according to preferred embodiments of the present invention include possibility to automatically measure movement variables such as speed, acceleration/retardation and/or direction of movement of a point or points of the moving body part, to use more than two points for measurement, to use more than one dynamic point for measurement, to automatically measure angles and areas and to use one-dimensional, two-dimensional and/or time resolved two-dimensional search fields for the automatically tracking of the dynamic point(s).

Still other objects and advantages of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should further be understood that the drawings are not necessarily drawn to scale and that, unless otherwise indicated, they are merely intended to conceptually illustrate the methods and systems herein.

### BRIEF DESCRIPTION OF THE DRAWINGS

In the following, the invention is described in greater detail applied by way of example to a heart. The description is made with reference to attached drawings, in which like reference characters denote similar elements and

FIG. 1 shows a composite of images in a dynamic sequence of schematic images of parts of a heart with a fixed reference point set in an apical part of the heart and a dynamic point set in a moving part of the left ventricular wall;

FIGS. 2a-d show separate images of the dynamic sequence shown in FIG. 1 and are also views of distance measurement;

FIGS. 3a-d show a first embodiment of the invention including the images shown in FIGS. 2a-d;

FIGS. 4a-d show a second embodiment of the invention including the images shown in FIGS. 2a-d;

FIGS. 5a-d show the images shown in FIGS. 2a-d, but with a second dynamic point set in the left ventricular wall;

FIGS. 6a-d are views of measurement of dynamic angles and dynamic areas in the images shown in FIGS. 5a-d;

FIGS. 7a-d show a fourth embodiment of the present invention including the images shown in FIGS. 5a-d;

FIGS. 8a-d show a fifth embodiment of the present invention including the images shown in FIGS. 5a-d;

FIGS. 9a-d show a sixth embodiment of the present invention including the images shown in FIGS. 5a-d;

FIG. 10 shows a schematic view of four points used for angiographic measurements.

### DESCRIPTION OF EMBODIMENTS

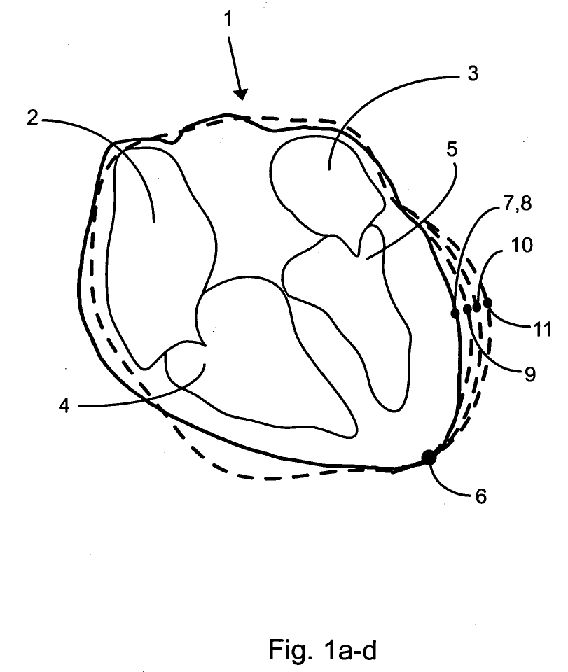

FIG. 1 shows a composite of highly schematic images of some parts of a heart 1. The images shown in FIG. 1 belong to a dynamic sequence of images of the heart 1, but are only a few images of a dynamic sequence covering a cardiac cycle of the heart 1. The sequence of images may be generated from projection data from time resolved two dimensional X-ray scanning of a portion of a body of a patient including the heart 1. The projection data may thus be time resolved two-dimensional data. In order to cover a complete cardiac cycle the images may be generated at an adequate frequency, for example 12,5 images per second. A contrast medium may be introduced before the scanning to visualize vessels properly on the images.

The images could be generated at any frequency that permits the analysis of the relevant moving body part. For example, the images could be generated at a rate of from about 9 images per second to about 24 images per second. However, the rate could be greater than about 24 images per second, particularly for fast moving body parts. On the other hand, the rate could be less than 9 images per second, particularly for slow moving body parts.

The heart 1 is a cyclically moving body part having a right atrium 2, a left atrium 3, a right ventricle 4 and a left ventricle 5. A single cardiac cycle consists of one diastolic phase (expansion phase) and one systolic phase (contraction phase) for the atria 2, 3 and one diastolic phase and one systolic phase for the ventricles 4, 5. The right ventricle 4 receives deoxygenated blood during its diastolic phase and pumps it into the pulmonary arteries during its systolic phase, while the left ventricle 5 receives oxygenated blood during its diastolic phase and pumps it into the aorta during its systolic phase. The heart muscles are supplied with blood by the coronary arteries, which encircle the heart and thus follow the expansion and contraction of the heart 1 during a cardiac cycle.

For different reasons it would be advantageous to be able to track a point of the heart 1 and measure for example movement variables of that point of the heart 1 relative a fixed point during the cardiac cycle. Movement variables, which might be interesting to measure may include distance, speed, acceleration, retardation, direction of movement of a point of the heart as well as dynamic angles and areas.

Using the method according to a preferred embodiment of the invention, a reference point 6 and a measurement point 7 typically are defined by a user. The reference point 6 may be set to a point that is fixed in its position relative the image geometry. That is, the reference point may be set to a point of a structure that is not essentially moved during a cardiac cycle. In the embodiment illustrated by the images shown in FIG. 1, the reference point 6 is set to an apical part of the heart 1, which is not essentially moved during a cardiac cycle. The reference point 6 is set in one image by a user and is then automatically indicated, that is, marked, in the other images in the sequence through image processing software.

In the embodiment illustrated by the images in FIG. 1, a point of the wall of the left ventricle 5 is desired to be measured and then that point is defined as the measurement point 7. The measurement point 7 may be a dynamic point, since it is moved during the cardiac cycle. The dynamic point 7 may also be set by a user in one image, and the corresponding points in the other images may then be automatically tracked by using image processing software including one of a number of known algorithms for automatic tracking of anatomic parts. An example of one such algorithm is FMI-SPOMF (Fourier-Mellin Invariant Symmetric Phase-Only Matched Filtering), described for instance by Qin-sheng Chen, Michel Defrise and F. Deconick in IEEE Transactions on Pattern Analysis and Machine Intelligence, Vol. 16, No. 12, pp 1146-1168, Dec. 1994. In order to improve the efficiency of the algorithm/algorithms used, the images may be pretreated to increase the contrasts between the imaged object and the background. The tracking may be performed using information about the imaged object and the darkest point may, for example, be tracked since it probably represents a vessel containing a lot of contrast medium.

The wall of the left ventricle 5 expands during the diastolic phase and contracts during the systolic phase. Thus, during the diastolic phase of the left ventricle 5, the dynamic point 7 typically moves from a position denoted 8 into expanded positions denoted 9, 10 and 11. The position 8 represents the end-systolic position of the point 7 and the position 11 represents the end-diastolic position of the point 7. Accordingly, during the systolic phase of the left ventricle 5, the point 7 may move from the position 11 into position 8.

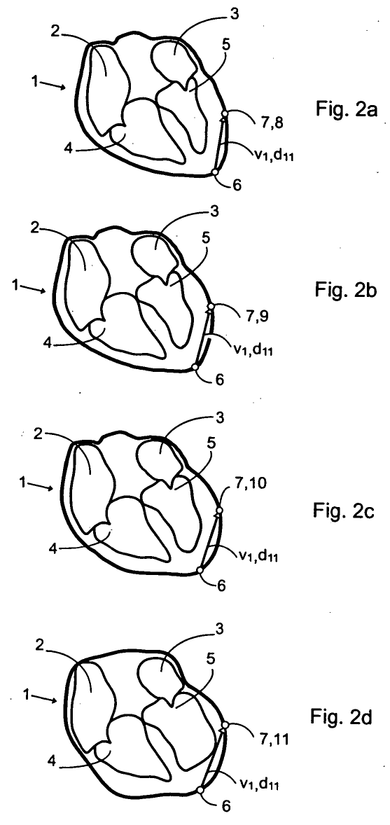

FIGS. 2a-d show separate images of the images in the dynamic sequence shown in FIG. 1 and are also views of distance measurement. The images in FIGS. 2a-d are subsequent in time. FIG. 2a shows an end-systolic view of the heart 1 and FIG. 2d an end-diastolic view of the heart 1. A length and a direction of a vector v₁ extending from the reference point 6 to the first dynamic point 7 may be automatically determined in all of the images of the sequence using image processing software. A first distance d₁₁ between the dynamic point 7 and the reference point 6 may also be automatically determined in each of the images in the sequence of images using the length of the vector v₁. The first distance d₁₁ between the reference point 6 and the dynamic point 7 may also be automatically compared between different images and used to determine the change of the position, i.e. the movement, of the dynamic point 7 during the cardiac cycle.

Since the data used for reconstruction of the images in FIG. 1 may be time resolved two-dimensional data, it may also be possible to determine a rate of change of the first distance d₁₁, i.e. movement variables, between different images in the sequence of images. The rate of change may also be automatically determined using image processing software.

Movement variables which may be determined may include for example speed, acceleration, retardation and direction of movement of the dynamic point 7 in different moments of the cardiac cycle, i.e. in different images of the sequence of images. Furthermore, the speed, acceleration and/or retardation with which the dynamic point 7 is moved between its different positions in two different images may also be determined. The movement variables may be measured in all of the below described embodiments of the present invention. When determining the rate of change of movement, the rate of change of any one or more of the variables may be determined.

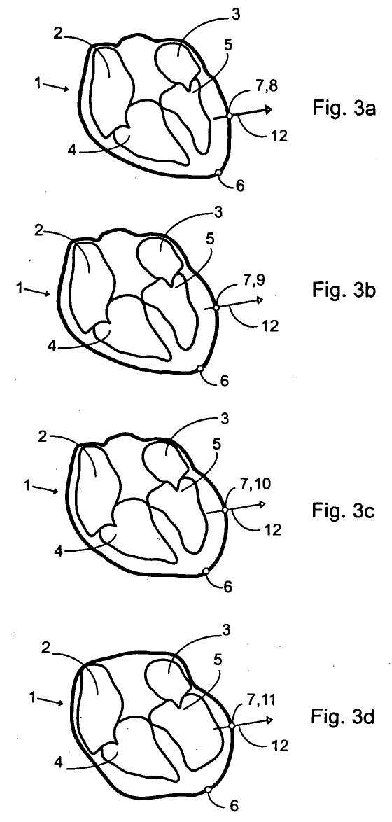

FIGS. 3a-d show a first embodiment of the invention including the images shown in FIGS. 2a-d. In the first embodiment of the invention a one-dimensional search field may be used for the tracking of the dynamic point 7 in the different images in the sequence of images. A vector 12, i.e. a line, representing the one-dimensional search field may be marked in one of the images and the dynamic point 7 may then be automatically tracked along that line 12 in all of the images in the sequence of images using image processing software as previously mentioned.

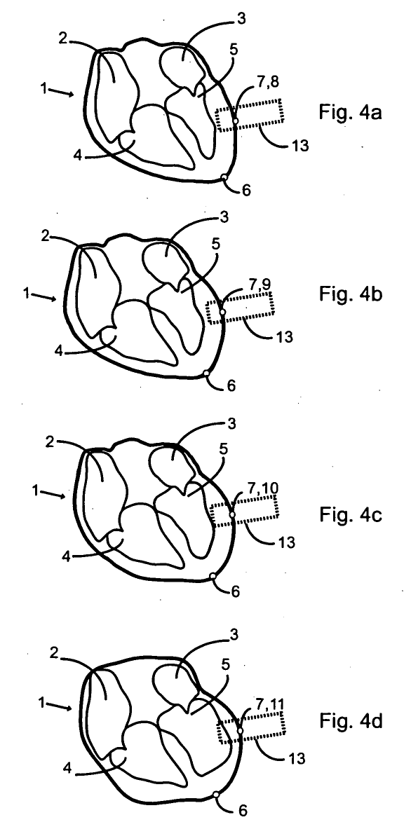

FIGS. 4a-d show a second embodiment of the invention including the images shown in FIGS. 2a-d. In the second embodiment of the invention a two-dimensional search field is used for the tracking of the dynamic point 7 in the different images in the sequence of images. An area 13, i.e. a rectangle, representing the two-dimensional search field may be marked in one of the images and the dynamic point 7 may then be automatically tracked within that rectangle 13 in all of the images in the sequence of images using image processing software as previously mentioned.

In a third embodiment (not shown) of the invention a time resolved two-dimensional search field may be used for the tracking of the dynamic point 7 in the different images in the sequence of images. An area 13, i.e. a rectangle, representing the two-dimensional search field may be marked in one of the images in the sequence of images and the dynamic point 7 may be automatically tracked within the rectangle using image processing software as previously mentioned, but information from previous and subsequent images may be used as guide to which point of possible points in the rectangle that is the searched dynamic point 7. Expected positions of the dynamic point 7, which may be based on information from previous images, may also be used to choose which point in the rectangle that is the searched dynamic point 7.

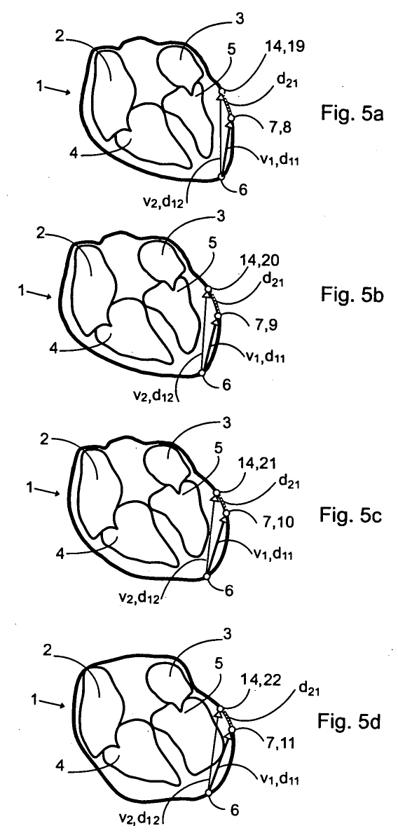

FIGS. 5a-d show the images shown in FIGS. 2a-d, but with a second dynamic point 14. Using the method according to the invention, movement of two different parts of the moving body part may simultaneously be measured by defining a second dynamic point 14, which may be different from the first dynamic point 7. In the FIGS. 5a-d, the second dynamic point 14 may be set to a point of the wall of the left ventricle 5 being different from the first dynamic point 7. The second dynamic point 14 may be moved from a position denoted 19 into expanded positions 20, 21 and 22. The position 19 may represent the end-systolic position of the point 14 and the position 22 may represent the end-diastolic position of the point 14. Accordingly, during the systolic phase of the left ventricle 5, the point 14 may be moved from position 22 into position 19. A length and a direction of the vector v₁ extending from the reference point 6 to the first dynamic point 7 as well as a vector v₂ extending from the reference point 6 to the second dynamic point 14 may then be automatically determined using image processing software.

The first distance d₁₁ between the first dynamic point 7 and the reference point 6 as well as the first distance d₁₂ between the second dynamic point 14 and the reference point 6 may then also be automatically determined using the length of the vector v₁ and the vector v₂, respectively, and compared between different images. Furthermore, the movement between the position in one image and the position in another image of both the first dynamic point 7 and the second dynamic point 14 as well as movement variables like, for example, the above mentioned may also be determined. It is also possible to automatically determine the movement of the two dynamic points 7, 14 relative each other through determining a second distance d₂₁ between the two dynamic points 7, 14 and comparing the second distance d₂₁ between different images. The second distance d₂₁ may be automatically determined using the lengths of the vectors v₁ and v₂ and using image processing software. Furthermore, using the two dynamic points 7, 14 it is also possible to measure dynamic angles and areas. Dynamic angles and areas may be measured in all of the below described embodiments.

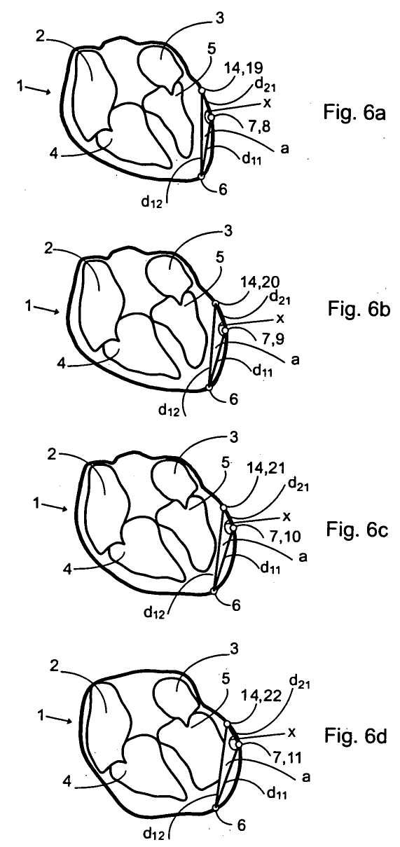

FIGS. 6a-d are views of measurement of dynamic angles and dynamic areas in the images shown in FIGS. 5a-d. A dynamic angle x may be automatically determined by using image processing software and by using the first distances d₁₁, d₁₂ and the second distance d₂₁ in each of the images of the sequence. Other angles than the shown angle x may of course also be determined. A dynamic area a may automatically be determined by using image processing software and by using the first distances d₁₁, d₁₂ and the second distance d₂₁ in each of the images of the sequence.

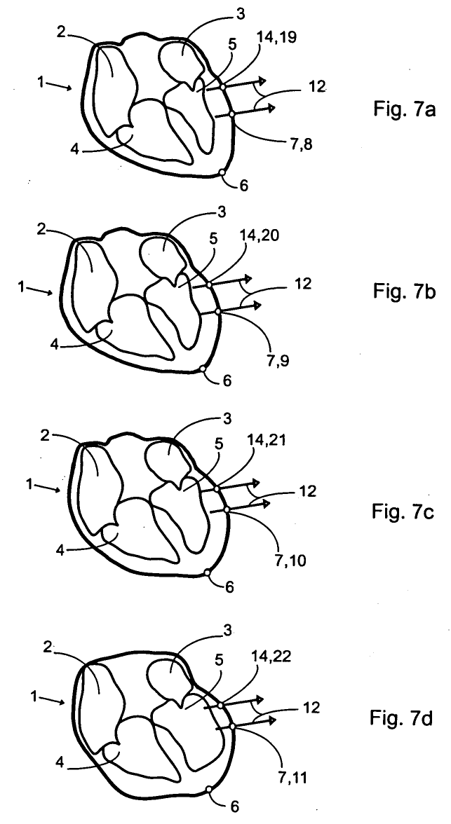

FIGS. 7a-d show a fourth embodiment of the invention including the images shown in FIGS. 5a-d. In the fourth embodiment of the invention one-dimensional search fields may be used for the tracking of the first and second dynamic points 7, 14, respectively, in the different images in the sequence of images. A vector 12, i.e. a line, representing the one-dimensional search field may be marked for each dynamic point 7, 14 in one of the images and the dynamic points 7, 14 may then be respectively tracked along the lines 12 in all of the images in the sequence of images using image processing software as previously mentioned.

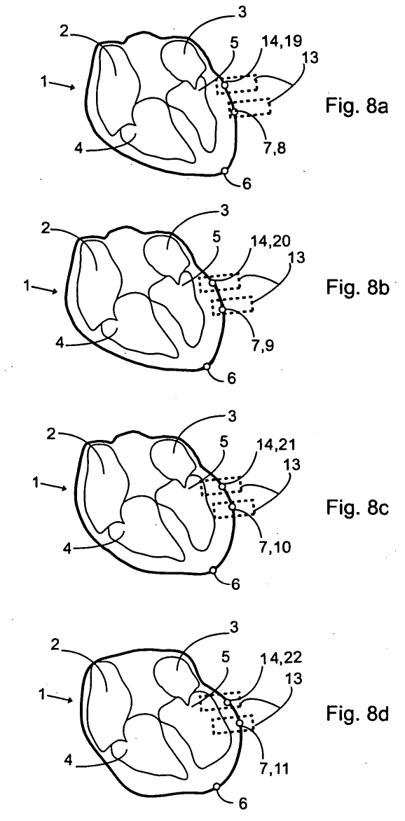

FIGS. 8a-d show a fifth embodiment of the invention including the images shown in FIGS. 5a-d. In the fifth embodiment of the invention two-dimensional search fields may be used for the tracking of the first and second dynamic points 7, 14, respectively, in the different images in the sequence of images. An area 13, i.e. a rectangle, representing the two-dimensional search field may be marked for each dynamic point 7, 14 in one of the images and the dynamic points 7, 14 may then be respectively tracked within those rectangles 13 in all of the images in the sequence of images using image processing software as previously mentioned.

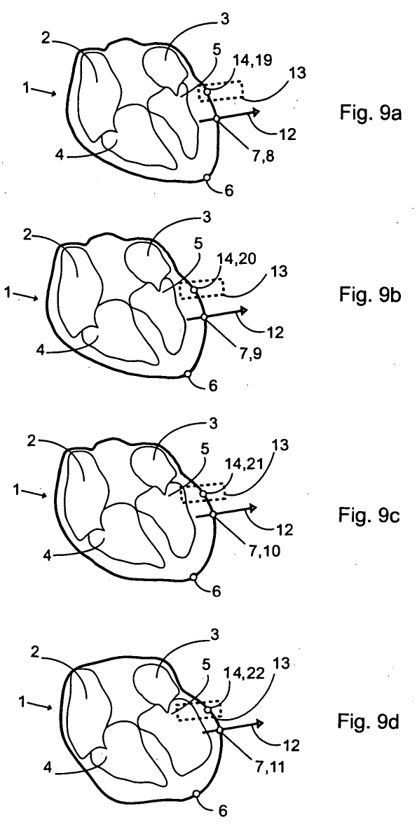

FIGS. 9a-d show a sixth embodiment of the invention including the images shown in FIGS. 5a-d. In the sixth embodiment of the invention a one-dimensional search field may be used for the tracking of one of the first and the second dynamic points 7, 14 and a two-dimensional search field may be used for the tracking of the other of the first and the second dynamic points 7, 14 in the different images in the sequence of images. A vector 12, i.e. a line representing the one-dimensional search field and an area 13, i.e. a rectangle, representing the two-dimensional search field may be marked in one of the images and the dynamic points 7, 14 may then be tracked along the line and within the rectangle, respectively, in all of the images in the sequence of images using image processing software as previously mentioned. In the FIGS. 9a-d a one-dimensional search field may be used for the tracking of the first dynamic point 7 and a two-dimensional search field may be used for the tracking of the second dynamic point 14, but instead a one-dimensional search field may be used for the tracking of the second dynamic point 14 and a two-dimensional search field may be used for the tracking of the first dynamic point 7.

In a seventh embodiment (not shown) of the invention time resolved two-dimensional search fields may be used for the tracking of the dynamic points 7, 14, respectively, in the different images in the sequence of images. An area 13, i.e. a rectangle, representing the two-dimensional search field may be marked for each dynamic point 7, 14 in one of the images in the sequence of images and the dynamic points 7, 14 may be respectively tracked within the rectangles using image processing software as previously mentioned, but information from previous and subsequent images is used as guide to which points of possible points in the rectangles that are the searched dynamic points 7, 14. Expected positions of the dynamic points 7, 14 based on information from previous images may also be used to choose which points that are the searched dynamic points 7, 14.

In an eighth embodiment (not shown) of the invention a one-dimensional search field may be used for the tracking of one of the first and the second dynamic points 7, 14 and a time resolved two-dimensional search field may be used for the tracking of the other of the first and the second dynamic point 7, 14 in the different images in the sequence of images. A vector 12, i.e. a line representing the one-dimensional search field and an area 13, i.e. a rectangle, representing the two-dimensional search field may be marked in one of the images and the dynamic points 7, 14 may then be respectively tracked along the line 12 and within the rectangle 13 in all of the images in the sequence of images using image processing software as previously mentioned. However, information from previous and subsequent images may be used as guide to which point of possible points in the rectangle 13 that may be the searched dynamic point 7, 14. Expected positions of the dynamic point 7, 14 based on information from previous images may also be used to choose which point in the rectangle that is the searched dynamic point 7, 14.

In a ninth embodiment (not shown) of the invention a two-dimensional search field may be used for the tracking of one of the first and the second dynamic points 7, 14 and a time resolved two-dimensional search field may be used for the tracking of the other of the first and the second dynamic points 7, 14 in the different images in the sequence of images. An area 13, i.e. a rectangle, representing the two-dimensional search field may be marked for each dynamic point 7, 14 in one of the images and the dynamic points 7, 14 may then be respectively tracked within that rectangles 13 in all of the images in the sequence of images using image processing software as previously mentioned. However, information from previous and subsequent images may be used as guide to which point of possible points in the rectangle 13 representing the time resolved two-dimensional search field that is the searched dynamic point 7, 14. Expected positions of the dynamic point 7, 14 based on information from previous images may also be used to choose which point in the rectangle 13 representing the time resolved two-dimensional search field that may be the searched dynamic point 7, 14.

The method according to the invention may be expanded to cover more than two dynamic points as well as more than one reference point. Any combinations of one-dimensional, two-dimensional and time resolved two-dimensional search fields for the tracking of the different points may then be possible to use.

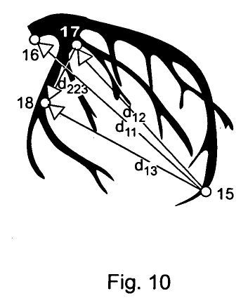

By way of example, an embodiment of the method according to the invention is described involving coronary angiography. Projection data of the coronary arteries are generated through coronary angiography, i.e. X-ray examination after injection of an opaque dye. The images are generated during at least one cardiac cycle at a frequency of 12.5 images per second. The right anterior oblique 30° projection has shown good results and is preferably used. A sequence of images may then be generated through reconstruction from the projection data. Four points 15, 16, 17, 18 may then be located and marked in one image in the sequence of images; a point 15 representing the most apical point of the coronary artery branches, a point 16 representing the lower contour of the left coronary ostium, a proximal point 17 and a distal point 18 on the horizontal part of the circumflex artery. The corresponding four points 15, 16, 17, 18 may then be automatically located in each of the images in the image sequence using image processing software. FIG. 10 shows a schematic view of the four points 15, 16, 17, 18 used for angiographic measurements.

The epicardial part of the apex is almost stationary during the heart cycle. Thus, the most apical point 15 of the coronary artery branches is almost stationary during the heart cycle and may therefore be used as a reference point. The other three previously mentioned points 16, 17, 18 may be dynamic points representing a first, a second and a third dynamic point. A one-dimensional, a two-dimensional or a time resolved two-dimensional search field may be used for the tracking of each of the corresponding dynamic points 16, 17, 18 in the other images in the sequence of images. The first distance d₁₁ from the most apical point 15 of the coronary artery branches to the point 16 representing the lower contour of the left coronary ostium may then be automatically determined in each image showing the amplitude of the left coronary ostium motion. The first distance d₁₂ between the most apical point 15 of the coronary artery branches and the proximal point 17 on the horizontal part of the circumflex artery as well as the first distance d₁₃ between the most apical point 15 of the coronary artery branches and the distal point 18 on the horizontal part of the circumflex artery may also be automatically determined in each image showing the motion of the proximal point 17 and the distal point 18, respectively. Furthermore, the second distance d₂₂₃ between the proximal point 17 and the distal point 18 of the circumflex artery may be determined, which second distance d₂₂₃ represents a portion of the circumflex artery extending along the atrioventricular groove. The motion of that portion of the circumflex artery roughly follows the most basal part of the left ventricular wall, which means that change of the distance between the proximal and the distal point of the circumflex artery represents the shortening and lengthening of the left ventricle 5.

Although cardiac imaging embodiments are described in detail, the invention has more general applicability and may be applied to another moving body part, such as for example the lungs. Furthermore, the method according to the invention may also be expanded to work on time resolved three-dimensional angiographies as well as simultaneous measuring of dynamic points in different body parts.

Thus, while there have been shown and described and pointed out fundamental novel features of the invention as applied to embodiments thereof, it will be understood that various omissions and substitutions and changes in details of the methods described, and in their operation, may be made by those skilled in the art without departing from the spirit of the invention. For example, it is expressly intended that all combinations of those method steps and/or system elements which perform substantially the same function in substantially the same way to achieve the same results are within the scope of the invention. Moreover, it should be recognized that method steps and/or system elements shown and/or described in connection with any disclosed form or embodiment of the invention may be incorporated in any other disclosed or described or suggested form or embodiment as a general matter of design choice.

1. A method for measuring in a dynamic sequence of medical images of a moving body part, the method comprising:
defining at least one measurement point in the moving body part in one of said images;
defining a reference point in one of said images to a point being fixed relative to an image geometry, said reference point being different from said at least one measurement point;
automatically tracking the at least one measurement point in all of said images of the sequence;
automatically indicating the reference point in all of said images of the sequence;
automatically determining a length and a direction of at least one vector extending from the reference point to one of the at least one measurement points for each pair of reference point and one measurement point in all of said images of the sequence, and
automatically determining at least one of a rate of change of said length and said direction of said at least one vector between selected images in said sequence of images.

2. The method according to claim 1, further comprising:
automatically determining at least one first distance between the reference point and one of the at least one measurement points for each pair of one reference point and one measurement point using the length of the corresponding vector.

3. The method according to claim 1, further comprising:
automatically determining a direction of movement of the at least one measurement point by using the direction of the corresponding vector.

4. The method according to claim 2, further comprising:
automatically determining a direction of movement of the at least one measurement point by using the direction of the corresponding vector.

5. The method according to claim 1, further comprising:
automatically determining a speed of the at least one measurement point by using said rate of change of the length of the corresponding vector.

6. The method according to claim 4, further comprising:
automatically determining a speed of the at least one measurement point by using said rate of change of the length of the corresponding vector.

7. The method according to claim 1, further comprising:
automatically determining at least one of an acceleration and a retardation of the at least one measurement point by using said rate of change of the length of the corresponding vector.

8. The method according to claim 6, further comprising:
automatically determining at least one of an acceleration and a retardation of the at least one measurement point by using said rate of change of the length of the corresponding vector.

9. The method according to claim 1, further comprising:
automatically comparing at least one of said rate of change of said length and said direction of said at least one vector between selected images in said sequence of images.

10. The method according to claim 2, further comprising:
automatically comparing said at least one first distance between selected images in said sequence of images.

11. The method according to claim 5, further comprising:
automatically comparing said speed between selected images in said sequence of images.

12. The method according to claim 7, further comprising:
automatically comparing the at least one of acceleration and retardation between selected images in said sequence of images.

13. The method according to claim 3, further comprising:
automatically comparing said direction of movement between selected images in said sequence of images.

14. The method according to claim 1, wherein the step of defining at least one measurement point in the moving body part in one of said images comprises defining at least two measurement points.

15. The method according to claim 14, further comprising:
automatically determining a second distance between two of the at least two measurement points for each pair of two measurement points using said lengths of the corresponding vectors.

16. The method according to claim 15, further comprising:
automatically comparing said second distance between selected images in said sequence of images.

17. The method according to claim 2, wherein the step of defining at least one measurement point in the moving body part in one of said images comprises defining at least two measurement points.

18. The method according to claim 17, further comprising:
automatically determining a second distance between two of the at least two measurement points for each pair of two measurement points using the first lengths of the corresponding vectors.

19. The method according to claim 18, further comprising:
determining at least one dynamic angle using at least one of said first distance and said second distance.

20. The method according to claim 18, further comprising:
determining an area using at least one of said first distance and said second distance.

21. The method according to claim 19, further comprising:
determining an area using at least one of said first distance and said second distance.

22. The method according to claim 1, wherein the step of automatically tracking the at least one measurement point is preceded by a step of creating in each of said images a one-dimensional search field for the tracking of each of the at least one measurement points.

23. The method according to claim 8, wherein the step of automatically tracking the at least one measurement point is preceded by a step of creating in each of said images a one-dimensional search field for the tracking of each of the at least one measurement points.

24. The method according to claim 1, wherein the step of automatically tracking the at least one measurement point is preceded by a step of creating in each of said images a two-dimensional search field for the tracking of each of the at least one measurement points.

25. The method according to claim 8, wherein the step of automatically tracking the at least one measurement point is preceded by a step of creating in each of said images a two-dimensional search field for the tracking of each of the at least one measurement points.

26. The method according to claim 1, wherein the step of automatically tracking the at least one measurement point is preceded by a step of creating in each of said images a time resolved two-dimensional search field for the tracking of each of the at least one measurement points.

27. The method according to claim 8, wherein the step of automatically tracking the at least one measurement point is preceded by a step of creating in each of said images a time resolved two-dimensional search field for the tracking of each of the at least one measurement points.

28. The method according to claim 26, wherein the step of creating in each of said images a time resolved two-dimensional search field for the tracking of the at least one measurement point further comprises creating the search field using information from at least one of previous and following images in said sequence of images.

29. The method according to claim 26, wherein the step of creating in each of said images a time resolved two-dimensional search field for the tracking of the at least one measurement point further comprises creating the search field using expected values based on information from the previous image in said sequence of images.

30. The method according to claim 14, wherein the step of automatically tracking the at least two measurement points is preceded by a step of creating in each of said images any combinations of one-dimensional search fields, two-dimensional search fields and time resolved two-dimensional search fields for the tracking of the at least two measurement points.

31. The method according to claim 1, wherein the step of automatically tracking the at least one measurement point is preceded by a step of image processing increasing the contrast between a reproduced object and a background.

32. The method according to claim 1, wherein the step of automatically tracking the at least one measurement point comprises using image processing software comprising at least one algorithm tracking the at least one measurement point.

33. A method for generating a dynamic sequence of medical images of a moving body part and measuring in said dynamic sequence, the method comprising:
scanning a portion of a body of a patient including the moving body part for generating time resolved projection data;
generating said images from said projection data;
defining at least one measurement point in the moving body part in one of said images;
defining a reference point in one of said images to a point being fixed relative to an image geometry, said reference point being different from said at least one measurement point;
automatically tracking the at least one measurement point in all of said images of the sequence;
automatically indicating the reference point in all of said images of the sequence;
automatically determining a length and a direction of at least one vector extending from the reference point to one of the at least one measurement points for each pair of reference point and one measurement point in all of said images of the sequence, and
automatically determining at least one of a rate of change of said length and said direction of said at least one vector between selected images in said sequence of images.

34. The method according to claim 33, further comprising:
automatically determining at least one first distance between the reference point and one of the at least one measurement points for each pair of one reference point and one measurement point using the length of the corresponding vector.

35. The method according to claim 33, further comprising:
automatically determining a direction of movement of the at least one measurement point by using the direction of the corresponding vector.

36. The method according to claim 34, further comprising:
automatically determining a direction of movement of the at least one measurement point by using the direction of the corresponding vector.

37. The method according to claim 33, further comprising:
automatically determining a speed of the at least one measurement point by using said rate of change of the length of the corresponding vector.

38. The method according to claim 36, further comprising:
automatically determining a speed of the at least one measurement point by using said rate of change of the length of the corresponding vector.

39. The method according to claim 33, further comprising:
automatically determining at least one of an acceleration and a retardation of the at least one measurement point by using said rate of change of the length of the corresponding vector.

40. The method according to claim 38, further comprising:
automatically determining at least one of an acceleration and a retardation of the at least one measurement point by using said rate of change of the length of the corresponding vector.

41. The method according to claim 33, further comprising:
automatically comparing at least one of said rate of change of said length and said direction of said at least one vector between selected images in said sequence of images.

42. The method according to claim 34, further comprising:
automatically comparing said at least one first distance between selected images in said sequence of images.

43. The method according to claim 37, further comprising:
automatically comparing said speed between selected images in said sequence of images.

44. The method according to claim 39, further comprising:
automatically comparing said at least one of acceleration and retardation between selected images in said sequence of images.

45. The method according to claim 35, further comprising:
automatically comparing said direction of movement between selected images in said sequence of images.

46. The method according to claim 33, wherein the step of defining at least one measurement point in the moving body part in one of said images comprises defining at least two measurement points.

47. The method according to claim 46, further comprising:
automatically determining a second distance between two of the at least two measurement points for each pair of two measurement points using said lengths of the corresponding vectors.

48. The method according to claim 47, further comprising:
automatically comparing said second distance between selected images in said sequence of images.

49. The method according to claim 34, wherein the step of defining at least one measurement point in the moving body part in one of said images comprises defining at least two measurement points.

50. The method according to claim 49, further comprising:
automatically determining a second distance between two of the at least two measurement points for each pair of two measurement points using the first lengths of the corresponding vectors.

51. The method according to claim 50, further comprising:
determining at least one dynamic angle using at least one of said first distance and said second distance.

52. The method according to claim 50, further comprising:
determining an area using at least one of said first distance and said second distance.

53. The method according to claim 51, further comprising:
determining an area using at least one of said first distance and said second distance.

54. The method according to claim 33, wherein the step of automatically tracking the at least one measurement point is preceded by a step of creating in each of said images a one-dimensional search field for the tracking of each of the at least one measurement points.

55. The method according to claim 40, wherein the step of automatically tracking the at least one measurement point is preceded by a step of creating in each of said images a one-dimensional search field for the tracking of each of the at least one measurement points.

56. The method according to claim 33, wherein the step of automatically tracking the at least one measurement point is preceded by a step of creating in each of said images a two-dimensional search field for the tracking of each of the at least one measurement points.

57. The method according to claim 40, wherein the step of automatically tracking the at least one measurement point is preceded by a step of creating in each of said images a two-dimensional search field for the tracking of each of the at least one measurement points.

58. The method according to claim 33, wherein the step of automatically tracking the at least one measurement point is preceded by a step of creating in each of said images a time resolved two-dimensional search field for the tracking of each of the at least one measurement points.

59. The method according to claim 40, wherein the step of automatically tracking the at least one measurement point is preceded by a step of creating in each of said images a time resolved two-dimensional search field for the tracking of each of the at least one measurement points.

60. The method according to claim 58, wherein the step of creating in each of said images a time resolved two-dimensional search field for the tracking of the at least one measurement point further comprises creating the search field using information from at least one of previous and following images in said sequence of images.

61. The method according to claim 58, wherein the step of creating in each of said images a time resolved two-dimensional search field for the tracking of the at least one measurement point further comprises creating the search field using expected values based on information from the previous image in said sequence of images.

62. The method according to claim 46, wherein the step of automatically tracking the at least two measurement points is preceded by a step of creating in each of said images any combinations of one-dimensional search fields, two-dimensional search fields and time resolved two-dimensional search fields for the tracking of the at least two measurement points.

63. The method according to claim 33, wherein the step of automatically tracking the at least one measurement point is preceded by a step of image processing increasing the contrast between a reproduced object and a background.

64. The method according to claim 33, wherein the step of automatically tracking the at least one measurement point comprises using image processing software comprising at least one algorithm tracking the at least one measurement point.

65. A system for measuring in a dynamic sequence of medical images of a moving body part, said system comprising means for:
defining at least one measurement point in the moving body part in one of said images;
defining a reference point in one of said images to a point being fixed relative to an image geometry, said reference point being different from said at least one measurement point;
automatically tracking the at least one measurement point in all of said images of the sequence;
automatically indicating the reference point in all of said images of the sequence;
automatically determining a length and a direction of at least one vector extending from the reference point to one of the at least one measurement points for each pair of reference point and one measurement point in all of said images of the sequence, and
automatically determining at least one of a rate of change of said length and said direction of said vector between selected images in said sequence of images.

66. A system for generating a dynamic sequence of medical images of a moving body part and for measuring in said dynamic sequence, said system comprising means for:
scanning a portion of a body of a patient including the moving body part for generating time resolved projection data;
generating said images from said projection data;
defining at least one measurement point in the moving body part in one of said images;
defining a reference point in one of said images to a point being fixed relative to an image geometry, said reference point being different from said at least one measurement point;
automatically tracking the at least one measurement point in all of said images of the sequence;
automatically indicating the reference point in all of said images of the sequence;
automatically determining a length and a direction of at least one vector extending from the reference point to one of the at least one measurement points for each pair of reference point and one measurement point in all of said images of the sequence and,
automatically determining at least one of a rate of change of said length and said direction of said at least one vector between selected images in said sequence of images.

67. The system according to claim 66, wherein the scanning is performed by a X-ray device.
